(19) United States
(12) Patent Application Publication
(10) Pub. No.: US 2021/0199852 A1
(43) Pub. Date: Jul. 1, 2021

(54) AUTOMATIC WATER SAMPLER APPARATUS

(71) Applicant: LUXEMBOURG INSTITUTE OF SCIENCE AND TECHNOLOGY (LIST), Esch-sur-Alzette (LU)

(72) Inventors: François BARNICH, Noville (BE); Nuria MARTINEZ-CARRERAS, Luxembourg (LU); Jean-François IFFLY, Hayange (FR); Oliver O'NAGY, Igel-Liersgberg (DE)

(21) Appl. No.: 16/757,691

(22) PCT Filed: Oct. 19, 2018
(86) PCT No.: PCT/EP2018/078773

§ 371 Date: Apr. 20, 2020

(30) Foreign Application Priority Data
Oct. 20, 2017 (LU) LU100486

(51) Int. Cl. G01W 1/14; G01N 1/18; G01N 33/18

(52) U.S. Cl. CPC G01W 1/14; G08B 21/12; G01N 33/18; G01N 1/18

(57) ABSTRACT

An automatic water sampler apparatus which comprises at least one movable injection unit that can be positioned in order to inject the sample in a specific vial disposed on a tray of the apparatus, the tray being on the base of the apparatus. The positioning is done by two positioning units. The input unit of the apparatus comprises at least one water collector, but can also comprise four or more water collectors. The invention is also directed to a method of sampling water from a predetermined water body, where a plurality of samples can be collected thanks to the automatic water sampler apparatus.

### CROSS-REFERENCE TO RELATED APPLICATIONS

The present invention is the US national stage under 35 U.S.C. § 371 of International Application No. PCT/EP2018/078773, which was filed on Oct. 19, 2018, and which claims the priority of application LU 100486 filed on Oct. 20, 2017, the content of which (text, drawings and claims) are incorporated here by reference in its entirety.

### FIELD

The present invention is directed to an automatic small-volume water sampler with a high collection capacity and to a method of sampling one or several predetermined water bodies.

### BACKGROUND

Over the past 30 years, geochemical and isotopic tracers have become common tools in hydrology. They have been key to decrypting the role of ‘pre-event’ water in stormflow response, ages of water in catchments, identifying water sources supporting runoff generation or plant-water uptake. Moreover, hydrological processes are highly dynamic in time and do often present nonlinear behaviour. Hence, high frequency data is needed to improve our mechanistic understanding of catchments.

Recently, progress in environmental monitoring and analytics has increasingly facilitated the collection of tracer data at high frequency (e.g. minutes), including nutrient concentrations (i.e. C, N, P), species (e.g. NO₃, NO₂, NH₄) and composition (e.g. dissolved organic matter, DOM). The review of Blaen P. J. et al., Sci Tot. Environ., 2016, 569-570, 647-660 describes the principles of in situ monitoring techniques (e.g. electrochemical detection, colorimetry, optical UV-Visible spectroscopy and optical fluorescence spectroscopy).

However, in situ analysers do not exist to measure the stable isotopes of oxygen and hydrogen in water, some major ions, and some parameters as phosphate and sulphate. Consequently, grab sampling in the field and subsequent analysis in the laboratory remain of major importance. Moreover, laboratory analysis of water samples collected in the field remains necessary to provide benchmarks against in situ instrument drift, cross-checks to detect unreliable readings, and backup measurements (Kirchner J. W. et al., Hydrol. Process., 2004, 18, 1353-1359).

Advances in high-sensitivity, multi-element analytical instruments such as ICP-MS have greatly reduced the sample volume required and routine analysis can be performed on millilitre sized samples (Chapin T. P. et al., Appl. Geochem., 2015, 59, 118-124). Hence, large volume samples are no longer needed. As described in this review about automated water samplers, an ideal water sampler would have the following attributes: small and easy to transport, low-cost, low-power, provide filtration and sample preservation, simple to deploy, capable of long-duration deployments and have a high sample capacity.

Nevertheless, an automatic water sampler having all of the above mentioned attributes plus being capable to collect water from different sources at the same time, is currently missing. In fact, all known samplers have one or more drawbacks, rendering high-frequency hydrology research a challenging and time-consuming task.

For instance, the “Siphon automatic sampler” from ISCO (see US patent application published U.S. Pat. No. 4,415,011) can collect samples from different water sources but has a limited storage capacity of 24 containers. The containers in this ISCO system have typically a volume ranging from 500 mL to 1000 mL, and sample preservation is not foreseen. Moreover, the ISCO sampler cannot collect samples from different sources in parallel.

A second example (Kim H. et al., Environ. Sci. TechnoL., 2012, 46, 11220-11226) is the “Siphon automatic sampler from ISCO coupled to a gravitational filtration system”. Again, although samples are filtered for a longer preservation, the storage capacity is limited to 24 containers and is thus not suited for high-frequency sampling campaigns.

Another example of liquid sampler has been described in the US patent application published US 2002/0025255 A1. The sampler was primarily designed for preserving samples of liquids containing volatile materials. It also comprises a refrigerator to cool the liquid and ensure sample preservation. However, its capacity is also limited to 24 containers.

An apparatus for the unattended collection of sequential, time-integrated water samples at pre-set time intervals has been disclosed in the granted US patent U.S. Pat. No. 7,687,028 B1. The water collector has a maximum storing capacity of 96 vials and can collect samples as small as 0.5 mL. Evaporation that could change the isotopic composition of the sample is minimized by sealing the opening of each sample vial by pressing each vial against a flat, low-friction sheet from the time each sample is filled until it is removed from the collector. Nonetheless, its capacity is also relatively low and the apparatus is not rinsed between samples to minimise contamination and memory effects.

As already stated, the major drawback of these systems is that they provide a storage capacity that is relatively low, that the volume of the containers or vials is too large for allowing direct analysis in the laboratory, and/or that the collectors are not designed for simultaneous collection of water coming from different sources.

### SUMMARY

The invention has for technical problem to alleviate at least one of the drawbacks present in the prior art.

The first object of the present invention is directed to an automatic water sampler apparatus comprising
a) an input unit adapted to collect water, the input unit comprising one or more of, or at least two of
   i. a first water collector for precipitation, the first water collector comprising a precipitation gauge,
   ii. a second water collector for surface water,
   iii. a third water collector for groundwater,
   iv. a fourth water collector for soil water,
b) one dosing unit per the water collector,
c) one movable injection unit, which comprises at least one needle, per the water collectors,
d) one tray with a plurality of vials, typically up to 1600 vials, the vials having in various instances a volume ranging from 1 mL to 200 mL, for example ranging from 2 mL to 40 mL,
e) an output unit per the water collector, adapted to evacuate the water outside the apparatus, and
f) a main controller unit adapted to control the input unit, the dosing unit, the movable injection unit and the output unit,
wherein each of the water collectors is independently in fluidic connection with the dosing unit and the movable injection unit,
wherein the dosing unit is in fluidic connection with the output unit, and wherein the main controller unit comprises a processor configured to perform:
   in the case of the input unit comprises one or more of the water collectors, sampling of the one or more of the water collectors, or
   in the case of the input unit comprises at least two of the water collectors, simultaneous sampling of the at least two of the water collectors.

According to an exemplary embodiment, the processor is configured to perform high-frequency sampling or high-frequency simultaneous sampling at a maximum rate ranging between one sample per minute and one sample per hour.

According to an exemplary embodiment, the processor is configured to perform sampling or simultaneous sampling at a rate ranging between one sample per minute and one sample per month.

According to an exemplary embodiment, the one dosing unit per the water collector comprises a reciprocating pump, in various instances a syringe.

According to an exemplary embodiment, the input unit comprises at least the first water collector for precipitation, the first water collector comprising:
a) a funnel adapted to collect precipitation,
b) a first closed container, and
c) a second closed container,
the first and second closed containers comprising respectively a first and a second air-release opening, a first and a second inlet and a first and a second outlet,
the first and second closed container being fluidly connected to the one dosing unit respectively through the first and second outlet by means of a merging element,
the funnel comprising a conduit, the conduit being fluidly connected to the first closed container through the first inlet.

According to an exemplary embodiment, the input unit comprises at least the first water collector for precipitation, the first water collector comprising:
a) a funnel adapted to collect precipitation,
b) a first closed container, and
c) a second closed container,
the first and second closed containers comprising respectively a first and a second air-release opening, a first and a second inlet and a first and a second outlet,
the first and second closed container being fluidly connected to the one dosing unit respectively through the first and second outlet by means of a merging element,
the funnel comprising a conduit, the conduit being fluidly connected to the second closed container through the second inlet.

According to an exemplary embodiment, the conduit is a flexible conduit.

According to an exemplary embodiment, the first and second closed container are fluidly connected to the one dosing unit respectively through the first and second outlet through a first and second 3-way stopcock.

According to an exemplary embodiment, each of the first and second 3-way stopcock comprises:
a) a first way which is respectively fluidly connected to the first outlet or the second outlet,
b) a second way which is fluidly connected to the one dosing unit by means of the merging element and
c) a third way, the third way being respectively a first fluidic exit and a second fluidic exit.

According to an exemplary embodiment, the first and second 3-way stopcock are electrically and/or mechanically connected to a first and second actuator, the first and second actuator comprising respectively a first and second control device, the first and second actuator being in various instances a first and second servomotor.

According to an exemplary embodiment, the funnel comprises a removable water filter.

According to an exemplary embodiment, the first and second closed containers each has a volume up to 500 mL, in various instances a volume up to 250 mL.

According to an exemplary embodiment, the fluidic connection between each of the water collectors and the corresponding dosing unit is a corresponding 4-way stopcock,
a) in the case wherein the input unit comprises at least the first water collector for precipitation, the 4-way stopcock comprises a first way which is fluidly connected to the merging element, and/or
in the case wherein the input unit comprises at least one of the second, third and fourth water collectors, the 4-way stopcock comprises a first way which is directly fluidly connected to the second, third or fourth water collectors,
b) the 4-way stopcock comprises a second way which is fluidly connected to the dosing unit, in various instances through a water filter,
c) the 4-way stopcock comprises a third way which is fluidly connected to the output unit, in various instances through a check valve, and
d) the 4-way stopcock comprises a fourth way which is fluidly connected to the movable injection unit.

According to an exemplary embodiment, the 4-way stopcock is electrically and/or mechanically connected to a third actuator, the third actuator comprising a third control device, the third actuator being in various instances a third servomotor.

According to an exemplary embodiment, the movable injection unit comprises two needles.

According to an exemplary embodiment, the apparatus is fitted within a frame, in various instances an aluminium frame, the frame further comprising two positioning units configured to position the at least one movable injection unit to a predefined location of the tray.

According to an exemplary embodiment, at the automatic water sampler apparatus further comprises at least one portable battery configured to power the automatic water sample apparatus.

According to an exemplary embodiment, the processor is a single-board computer, in various instances a Raspberry Pi.

According to an exemplary embodiment, the input unit comprises any combination of
a) at least one first water collector for precipitation,
b) at least one second water collector for surface water,
c) at least one third water collector for groundwater, and/or
d) at least one fourth water collector for soil water.

The second object of the present invention is directed to a method of sampling water from a predetermined water body, the method comprising the step of
a) providing an automatic water sampler apparatus to sample a water body,
b) rinsing the automatic water sampler apparatus with the water from the water body, and
c) collecting a plurality of samples of the water.

The method is remarkable in that the automatic water sampler apparatus is an automatic water sampler apparatus in accordance with the first object of the present invention.

According to an exemplary embodiment, a plurality of samples comprises an amount up to 1600 samples.

The invention is particularly interesting in that it provides an automatic water sampler apparatus which is capable of sampling water coming from several sources simultaneously.

The quantity of samples that can be collected is very high (up to 1600 vials) and samples are directly stored in vials compatible with analysis device(s) in the lab, reducing pre-treatment time and cost.

The sampling frequency and the sample volumes can be controlled.

The design of the apparatus is simple and it has a low energy consumption.

The method uses different conduits for each water source, minimising contamination and memory effects.

Samples are filtered for a longer preservation and sealed to prevent evaporation.

The water sampler apparatus can be programmed and remotely controlled.

The invention is portable and will allow collecting high frequency data in remote places.

The invention will further facilitate water sampling and make available extensive and unique water chemistry data sets for environmental monitoring agencies, wastewater treatment plants, hydrologists (scientists) and drinking water firms, among others. Newly gained data might lead to new insights into long-term water chemistry and pollution patterns and trends, and short-term dynamics of hydrological systems. Furthermore, newly gained data might have a valuable impact on water monitoring, policy and treatment, in natural or artificial environments.

### DRAWINGS

FIG. 1 is an exemplary representation of the water sampler apparatus, with two injection units, in accordance with various embodiments of the present invention.

FIG. 2 is an exemplary representation of the water sample apparatus, with four injection units, in accordance with various embodiments of the present invention.

FIG. 3 is an exemplary schematic representation of the sampling system of the water sampler apparatus, in accordance with various embodiments of the present invention.

FIG. 4 is an exemplary representation of the external precipitation collector, in accordance with various embodiments of the present invention.

FIG. 5 is an exemplary representation of junction between the external precipitation collector and the 4-way stopcock, in accordance with various embodiments of the present invention.

FIG. 6 is an exemplary representation of the 4-way stopcock functioning, in accordance with various embodiments of the present invention.

FIG. 7 is an exemplary analysis of the memory effects, in accordance with various embodiments of the present invention.

### DETAILED DESCRIPTION

The present invention is directed to an apparatus for the unattended/automatic sampling of water. The sampling of water includes time-integrated precipitation (rain, snow, hail . . . ) and punctual samples from different origins (surface water/stream water, groundwater, soil water, water from water treatment plants, water or wastewater from sewage treatment plants . . . ).

The invention uses a mechanical system to transfer a water sample into a sample vial, as it is already known (see Chapin T. P., Appl. Geochem., 2015, 59, 118-124).

The automatic sampler apparatus 100 allows water collection for the analysis of its properties, notably of the stable isotopes of oxygen and hydrogen in water.

The following will describe how the apparatus has been designed.

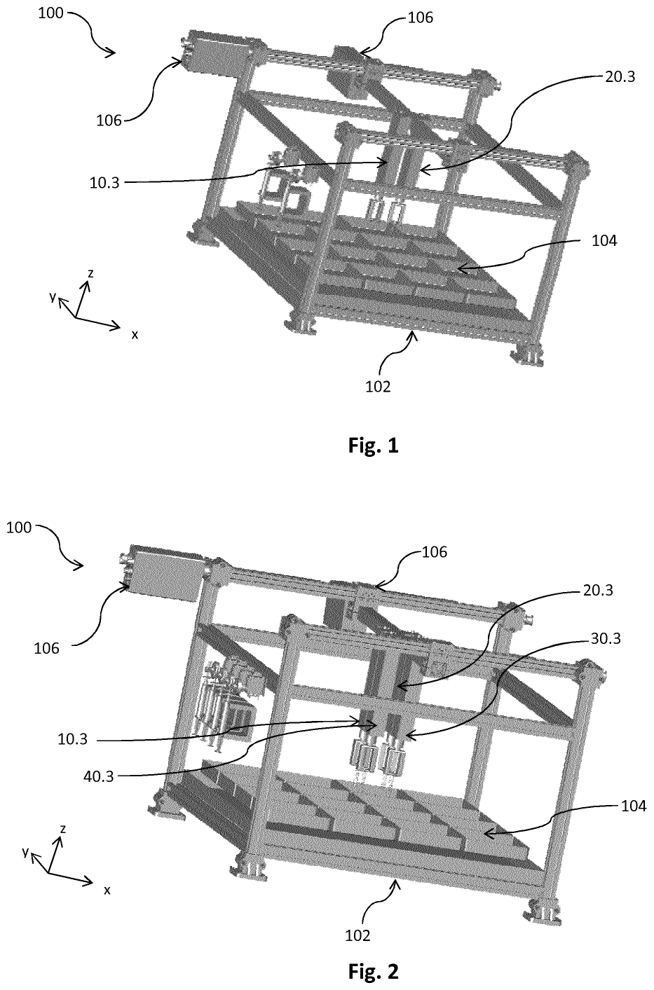

As shown in FIG. 1 or 2, the automatic sample apparatus 100 has a generally rectangular base 102 on which a tray 104 with a plurality of vials (not shown), typically up to 1600 vials, is disposed. The base 102 confers the generally cuboid shape of the apparatus 100.

The tray 104 can contain several standard laboratory storage boxes (for instance, on FIG. 1 or 2, 16 boxes are represented).

The tray 104 can be connected to a cooling system, in order to preserve samples from a potential degradation.

The volume of the vials can be comprised between 1 mL and 200 mL, in various instances between 2 mL and 40 mL. The vials can be directly used for laboratory experiments/analysis.

FIG. 1 also shows two positioning units (106) configured to move two injection units (10.3, 20.3) (only two injection units are represented on FIG. 1, but this is one of the possible examples of the apparatus 100 of the present invention, FIG. 2 is a view schematically showing another example with four injection units 10.3, 20.3, 30.3, 40.3).

Those injection units (10.3, 20.3) thus move in relation with a predefined location on the tray 104, more particularly in relation with a predefined vial.

An injection unit comprises at least one needle (not shown on FIG. 1, nor on FIG. 2), configured to penetrate the cap of the vials, which is usually a septum (in order to prevent evaporation of the sample as well as its potential contamination). The injection unit (10.3, 20.3, 30.3, 40.3) can comprise a second needle, the second needle being configured to penetrate the vials (as the first needle) in order to release the pressure in the vials when the other needle delivers the sample into the vial.

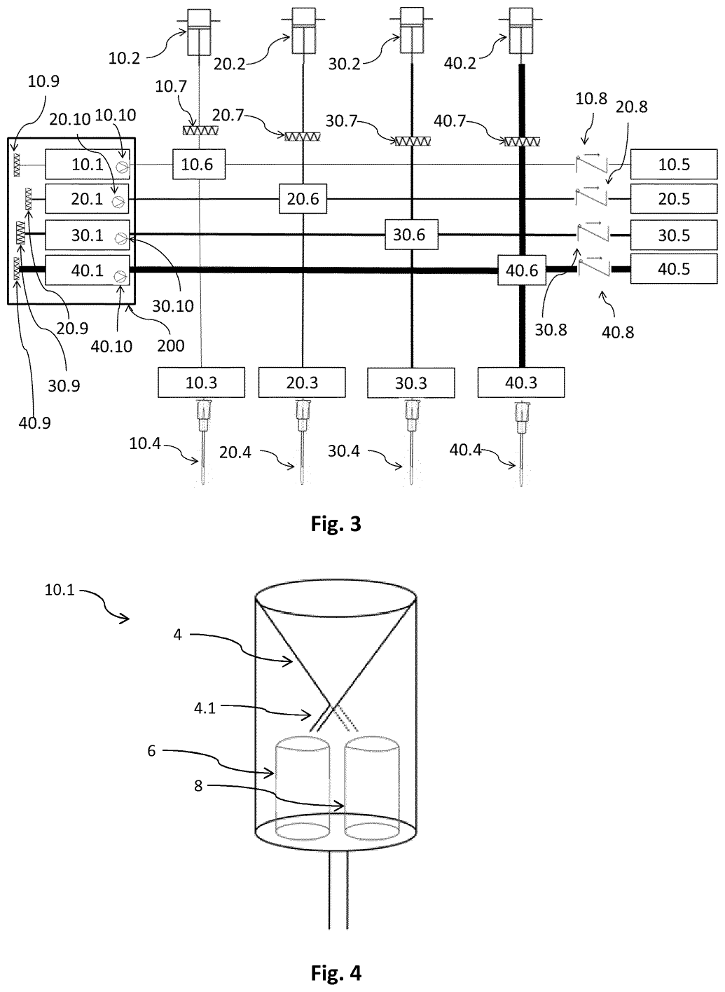

The injection unit is in fluidic connection with a corresponding input unit 200 configured to collect the water (see FIG. 3). The input unit 200 can comprise several water collectors to simultaneously sample different water types, in various instances a first water collector 10.1 for the precipitation, a second water collector 20.1 for surface water/stream water, a third water collector 30.1 for the ground water and/or a fourth water collector 40.1 for soil water.

The input unit 200 can also comprise two or more water collectors to sample the same type of water. The input unit 200 can also comprise more than four water collectors.

There is also a corresponding dosing unit (10.2, 20.2, 30.2, 40.2), which can comprise a reciprocating pump, for example a syringe. The syringe's cylindrical tube forming the syringe can be graduated. The volume capacity of the syringe can be comprised between 1 and 250 mL, in various instances amounting to 60 mL.

A corresponding output unit is finally added (10.5, 20.5, 30.5, 40.5), for water evacuation.

All these units are in fluidic connection together, as shown on the schematic representation of the sampling system of the water sampler apparatus (FIG. 3).

All the internal pieces, conduits, tubes and stopcocks in contact with water are laboratory dispensers of common use that can be easily replaced. Conduits are made of inert material (e.g. Teflon). This renders the system cheap, easy to manufacture and suitable for outdoor use.

All parts, excluding the precipitation collector 10.1, are sheltered inside a housing (not shown) that protects the water sampler apparatus 100 from environmental disturbance (e.g. rain, hail, snow, temperature variation, wind . . . ). The box has an opening that ensures the easy access to load and unload the storing boxes with the sampling containers. The storing boxes are locked on the rectangular base 102 of the water sampler apparatus 100. The apparatus 100 has a weight and a size that makes it portable.

The length of the apparatus 100 can be comprised between 80 cm and 160 cm, in various instances between 100 cm and 140 cm. For instance the length is equal to 120 cm.

The width of the apparatus 100 can be comprised between 80 cm and 160 cm, in various instances between 100 cm and 140 cm. For instance, the width is equal to 120 cm.

The height of the apparatus 100 can be comprised between 60 cm and 140 cm, in various instances between 80 cm and 120 cm. For instance, the height is equal to 100 cm.

The weight of the apparatus can be comprised between 40 kg and 100 kg. The weight of the apparatus is for instance equal to 80 kg. In all cases, the apparatus 100 is light enough to be transportable.

### The Input Unit 200

The input unit 200 is adapted to collect the water that is to be introduced inside the vials.

The input unit 200 can comprise a first water collector 10.1, which is used to collect precipitation (rain, snow, hail . . . ). In this case, the first water collector 10.1 comprises a precipitation gauge (or pluviometer) (not shown), that is necessary to detect the occurrence of precipitation as well as its amount. A precipitation sensor might also be added (in this case, the amount of precipitation may not be measured). Once the occurrence of a precipitation is detected and/or measured, the main controller unit may trigger (or may not trigger) the precipitation sampling (according to the sampling scheme determined by the user).

The input unit 200 can comprise a second water collector 20.1, which is used to collect surface water and/or stream water.

The input unit 200 can comprise a third water collector 30.1, which is used to collect groundwater.

The input unit 200 can comprise a fourth water collector 40.1, which is used to collect soil water.

The input unit 200 can comprise additional water collectors (not represented).

The input unit 200 can comprise any combination of:
a) at least one first water collector 10.1 for precipitation,
b) at least one second water collector 20.1 for surface water,
c) at least one third water collector 30.1 for groundwater, and/or
d) at least one fourth water collector 40.1 for soil water.

Thus, the input unit 200 can have for instance two or more first water collector 10.1 and none of the other type of collectors.

The input unit 200 comprises several pumps, necessary to direct the flow of water from the sampling point through the apparatus 100 and towards the output unit (10.5, 20.5, 30.5, 40.5) of the apparatus 100. More particularly, the first water collector 10.1 comprises a pump 10.10, the second water collector 20.1 comprises a pump 20.10, the third water collector 30.1 comprises a pump 30.10 and the fourth water collector 40.1 comprises a pump 40.10. In case where additional water collectors are mounted in the apparatus 100, each of the additional water collectors would also comprise a pump.

In the case of the first water collector 10.1, adapted to collect precipitation, the pump 10.10 is actually two pumps (see below).

On the drawing of FIG. 1, representing the automatic water sampler apparatus 100 with only two injection units (e.g. 10.3, 20.3), the input unit 200 of the represented apparatus then only comprises two water collectors. Similarly, on the drawing of FIG. 2, representing the automatic water sampler apparatus 100 with four injection units (10.3, 20.3, 30.3, 40.3), the input unit 200 of the represented apparatus then comprises four water collectors.

An important advantage of the water sampler apparatus of the present invention is that the main controller unit, via its processor, can trigger the sampling of all the water collectors in a simultaneous way.

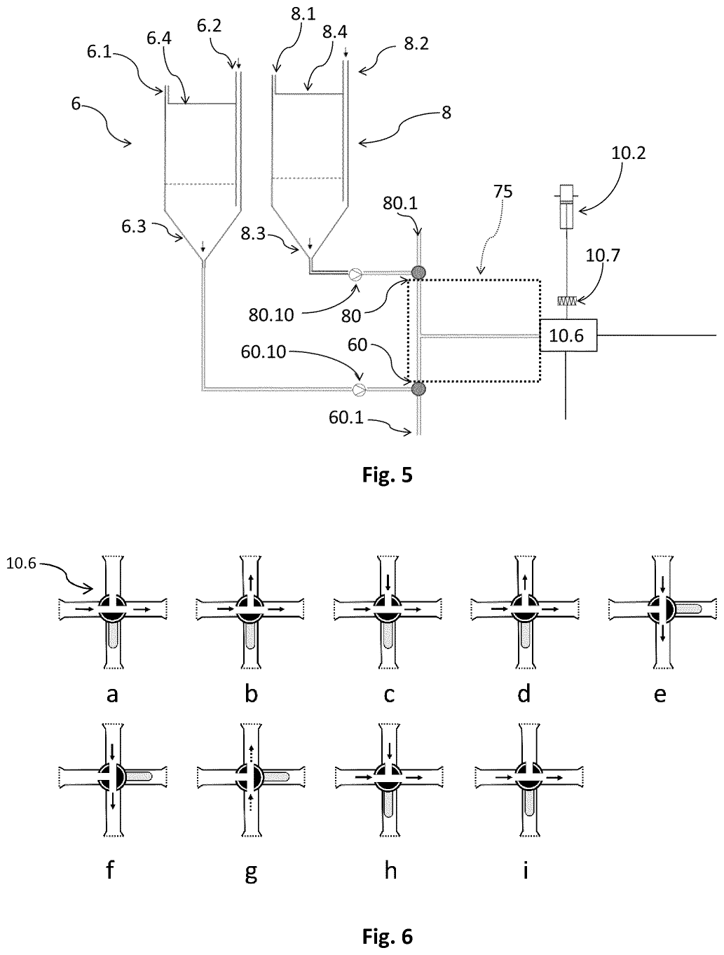

The external precipitation collector (FIGS. 4 and 5)

The first water collector 10.1, or the external precipitation collector, which is used to collect precipitation is schematically represented on FIG. 4. The collector 10.1 comprises a funnel 4 (by which precipitation is collected), a first closed container 6 and a second closed container 8. The closed containers (6, 8) can be bottles.

The first closed container 6 and second closed container 8 are identical to each other and, as represented on FIG. 5, they are in connection with the rest of the water sampler apparatus 100.

Each of the closed containers (6, 8) comprises three openings: an air-release opening (6.1, 8.1) to evacuate the excessive pressure, an inlet (6.2, 8.2) and an outlet (6.3, 8.3). The inlet (6.2, 8.2) of the closed container is used to fluidly connect the container (6, 8) to the funnel 4 by means of a conduit 4.1. The outlet (6.3, 8.3) of the closed container (6, 8) is used to fluidly connect the container (6, 8) to the corresponding dosing unit 10.2.

The precipitation collector 10.1 collects time-integrated samples. The main controller unit of the water sampler controls the operation of the precipitation sampler. It is the main controller unit that receives signals from the precipitation gauge and initiates precipitation sampling. Hence, the precipitation sampler does not function independently, but needs the input signal from a precipitation gauge or a rain sensor.

Precipitation falls inside the funnel 4 and passes through a removable water filter 10.9 (shown on FIG. 3), namely a removable sieve that traps litter or suspended particles. This is to prevent clogging of the conduits.

The sieve mesh size is rather coarse. It can be comprised between 0.5 mm and 5 mm, in various instances with a size of 2 mm. In fact, the sieve aims at preventing “big” litter, such as leaves or stones, from entering into the water sampler apparatus.

The funnel 4 is in various instances built in a material that reduces the retention of water. It can also be built in aluminum and linked to a thermostat and a heater that melts solid precipitation (snow, hail . . . ). However, the latest will increase the energy consumption, reduce portability and enhance fractionation of the oxygen and hydrogen isotopes of water. The size of the funnel 4 can vary according to the expected precipitation (e.g. intensity) and the amount of sample to be collected in different climatic conditions and/or sampling periods.

The bottom of the funnel 4 is connected to a short flexible conduit 4.1 that allows precipitation to directly flow by gravity into (a) the first container 6, (b) the second container 8, or (c) to be directed outside the precipitation sampler for its removal.

A mechanical placement device (not shown) moves the conduit 4.1 between the three positions. On FIG. 4, the conduit 4.1 is in fluidic connection with the first container 6. The dashed lines of FIG. 4 indicate that the conduit 4.1 can also be in fluidic connection with the second container 8. When the sampler is not activated by the main controller unit, water falling into the funnel 4 is always directed outside the sampler. Once activated, precipitation flows into one of the containers (6, 8) for homogenisation during a predetermine time interval or after cumulating a certain precipitation volume. When the sampling interval is over, the controller directs the placement device to the other container, which might be filled in by precipitation, or outside the sampler. The placement device moves the flexible conduit 4.1 between positions within a few seconds, preventing water loss and sample mixing.

When precipitation is collected, the water ends in one of the container thanks through the conduit 4.1 and is then directed, through the outlet 6.3 of the first container 6 or through the outlet 8.3 of the second container 8, to the dosing unit 10.2 or to the output unit 10.5.

A first 3-way stopcock 60 directs water stored in the first closed container 6 into the dosing unit 10.2 or into the output unit 10.5.

A second 3-way stopcock 80 directs water stored in the second closed container 8 into the dosing unit 10.2 or into the output unit 10.5.

A merging element 75, in various instances a pipe tee or a Y-shape connector, is fluidly connected to the first and second 3-way stopcock (60, 80) and to the dosing unit 10.2 and output unit 10.5 via a 4-way stopcock 10.6 (see details below).

When precipitation is sampled by the dosing unit 10.2, the first 3-way stopcock 60 lets the water from the first closed container 6 flow through the merging element 75 in the 4-way stopcock 10.6, thanks to the inflow directed by the pump 60.10, while precipitation is simultaneously collected in the second closed container 8 and vice versa. In other words, the water from the second closed container 8 can also flow through the merging element 75 toward the 4-way stopcock 10.6, thanks to the inflow directed by the pump 80.10, while precipitation is simultaneously collected in the first closed container 6.

Both pumps (60.10, 80.10) on FIG. 5 are schematically equivalent to the pump 10.10 of FIG. 3.

The 4-way stopcock 10.6 is in fact the fluidic connection between the precipitation collector 10.1 and the corresponding dosing unit 10.2.

A fluidic exit (60.1, 80.1) is present in both 3-way stopcocks (60, 80) to allow water evacuation from the system. For instance, water can be evacuated (instead of sampling) when the first and second closed containers (6, 8) are full due to heavy precipitation or need to be cleaned. Alternatively, if the user is not interested in sampling precipitation, there is still a need to empty the precipitation container if it contains water.

Both 3-way stopcocks (60, 80) are each electrically and/or mechanically connected to an actuator with a control device, controlled by the main controller unit. The actuator can be a servomotor, such kind of actuator allowing for precise control of angular or linear position, velocity and acceleration.

A possible scheme would pump enough precipitation to rinse the 3-way stopcocks (60, 80), the 4-way stopcock 10.6, the dosing unit 10.2 (with the syringe) and the injection unit 10.3 in order to prevent any contamination or memory effect. In other words, the water sampler apparatus 100, in particular the line pertaining to the first water collector, is washed with the water that is going to be sampled thereafter.

The volume of the precipitation containers (6, 8) (i.e. the first and second container) can vary (e.g. up to 500 mL, in various instances of 250 mL). These closed containers (6, 8) are designed to collect time—or volume—integrated samples and avoid evaporation during the sampling period. To this end, it is designed to reduce the surface of water in contact with air. Precipitation falling from the funnel 4 flows into the container through a conduit 4.1 down to the bottom of the container. Only a conduit 4.1 with a small internal diameter and located in the upper part of the container allows adjusting the air pressure inside the container.

The closed containers (6, 8) have in various instances a special shape in order to be able to deal with small and large precipitation volumes. Precipitation falling in the container will first fill in the bottom part of the container, with a conic shape and a smaller lower diameter, and then the upper part, with a larger diameter.

The container (6, 8) is in fact closed with a cap (6.4, 8.4). However, it is possible that a mobile and floating plastic piece, with a diameter amounting to the inner diameter of the largest part of the container, stands inside the container and move when the water is rising. It is aimed at sealing the container against evaporation.

The funnel 4 and the containers (6, 8) of the precipitation collector 10.1 are protected inside an insulation cover, in various instances opaque in order to protect the samplings from the UV irradiation, especially from the sun, which, in turn, should be mounted on a mast following standard rain gauge installation guidelines.

### The Other Water Collectors

The automatic water sampler apparatus 100 can comprise a second water collector 20.1 which is adapted for sampling surface water/stream water, a third water collector 30.1 which is adapted for sampling groundwater and a fourth water collector 40.1 which is adapted for sampling soil water. Additional water collectors (not shown) can be connected to the water sampler apparatus.

As the precipitation water collector 10.1, each of the other water collectors is independently in fluidic connection with a corresponding dosing unit, injection unit and/or output unit.

A coarse water filter (20.9, 30.9, 40.9) can be placed upstream of the second, third and fourth water collectors in order to allow for the removal of suspended particles or litter and to prevent clogging of the conduits. Those filters (20.9, 30.9, 40.9) have a similar function as the filter 10.9 used in the external precipitation collector 10.1 and have subsequently the same features in terms of mesh size.

As stated above, each of the second, third and fourth water collector (20.1, 30.1, 40.1) comprises a pump (20.10, 30.10, 40.10), which is used for controlling the inflow of water to be analysed through the system and toward the corresponding output unit (20.5, 30.5, 40.5).

### The Dosing Units

Each of the water collectors (10.1, 20.1, 30.1, 40.1) is in fluidic connection with one corresponding dosing unit (10.2, 20.2, 30.2, 40.2). Therefore, in the example of FIG. 1, two dosing units are present, since there are only two water collectors. Similarly, in the example of FIG. 2, four dosing units are present and are fluidly connected with the corresponding four water collectors. One dosing unit can comprise a reciprocating pump. An example of reciprocating pump is a syringe, which can hold a volume ranging from 1 mL to 250 mL and which can reinject the sample on demand.

The fluidic connection between each of the water collectors (10.1, 20.1, 30.1, 40.1) and the corresponding dosing unit (10.2, 20.2, 30.2, 40.2) is a 4-way stopcock (10.6, 20.6, 30.6, 40.6), as schematically shown on FIG. 5. The 4-way stopcock (10.6, 20.6, 30.6, 40.6) also fluidly connects the system with the injection unit (10.3, 20.3, 30.3, 40.3) and the output unit (10.5, 20.5, 30.5, 40.5).

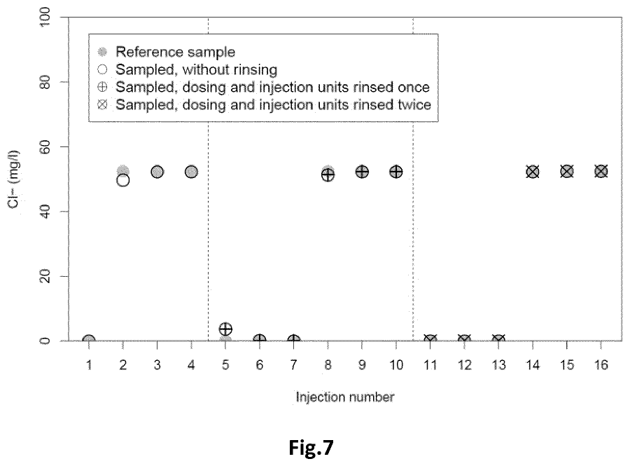

The 4-way stopcock (10.6, 20.6, 30.6, 40.6) is designed to control the flow of a liquid. It is chemically resistant and can be constructed in different materials (e.g. polycarbonate). It consists of a housing where liquid flows and a cork that is fitted inside the housing. To this end, the cork also consists of an external handle (the black circle on FIG. 6) that allows changing the flow paths in relation to the four ways. It is also possible to close the fluid paths by turning the stopcock handle to an intermediate position. The operation is motor driven and controlled by the main controller unit.

### With an External Precipitation Collector

With an external precipitation collector 10.1, the 4-way stopcock 10.6 is fluidly connected to the first and second 3-way stopcocks (60, 80) via the merging element 75 which directs water stored in one of the closed container (6, 8) into the dosing unit 10.2. This is done through a first way of the 4-way stopcock 10.6.

A second way of the 4-way stopcock 10.6 is fluidly connected to the corresponding dosing unit 10.2, or the syringe, in various instances through a corresponding water filter 10.7 to allow removing the remaining particles that might have crossed the coarser water filter.

The water filter 10.7 is thus finer than the water filter 10.9. The water filter 10.7 has a pore size comprised between 0.300 μm and 10 μm. For example, the water filter 10.7 has a pore size of 5 μm.

A third way of the 4-way stopcock 10.6 is fluidly connected to the corresponding output unit 10.5 of the water sampler apparatus, in various instances through a corresponding check valve 10.8.

A fourth way of the 4-way stopcock 10.6 is fluidly connected to a corresponding injection unit 10.3, which comprises at least one corresponding needle 10.4.

### With the Other Water Collectors

With the other water collectors (20.1, 30.1, 40.1), the corresponding 4-way stopcock (20.6, 30.6, 40.6), and in particular a first way, is directly fluidly connected to the corresponding water collectors, in various instances via a tubing or a conduit. The tubing or conduit is in various instances flexible.

A second way of the 4-way stopcock (20.6, 30.6, 40.6) is fluidly connected to the corresponding dosing unit (20.2, 30.2, 40.2), or the syringe, in various instances through a corresponding water filter (20.7, 30.7, 40.7) to allow removing the remaining particles that might have crossed the coarser water filter.

The water filters (20.7, 30.7, 40.7) have pore sizes identical to the water filter 10.7.

A third way of the 4-way stopcock (20.6, 30.6, 40.6) is fluidly connected to the corresponding output unit (20.5, 30.5, 40.5) of the water sampler apparatus, in various instances through a corresponding check valve (20.8, 30.8, 40.8).

A fourth way of the 4-way stopcock (20.6, 30.6, 40.6) is fluidly connected to a corresponding injection unit (20.3, 30.3, 40.3), which comprises at least one corresponding needle (20.4, 30.4, 40.4).

All the 4-way stopcocks (10.6, 20.6, 30.6, 40.6) are electrically and/or mechanically connected to an actuator comprising a control device, the actuator being a servomotor. The main controller unit is controlling the actuator, such kind of actuator allowing for precise control of angular or linear position, velocity and acceleration.

### The Injection Units

The injection unit (10.3, 20.3, 30.3, 40.3) sustains a needle (10.4, 20.4, 30.4, 40.4) for each water type being sampled. During water sampling, the injection unit, reaches the exact position for delivering the sample to a predetermined vial. Alternatively, the injection unit can move to a ‘trash’ reservoir or to a position where it can be directly evacuated from the sampler (notably in case of rinsing). It is the main controller unit that defines the x-y position (see on FIG. 1) to be reached and activates the motor controllers with absolute positioning sensing that will operate the linear motor drivers. Once the position is reached and the sampling volume is ready to fill in the vial, the main controller unit will move the needle up and down (z direction) for delivering the sample into the predetermined vial.

The injection unit (10.3, 20.3, 30.3, 40.3) is sustained by a frame, in various instances build in aluminium because of its lightness.

Two linear positioning units, with an integrated motor controller, are mounted on the frame and they ensure motion in the x-y plane. The system allows the movement of the injection unit to a very specific location, i.e. the pre-defined vial position. Movement and exact coordinates (x-y) are determined by the main controller unit. The x-y positioning works with an absolute position sensing system, that allows a complete system shut down without losing information about the position. The position resolution is 3 mm/1000 counts.

The injection unit comprises at least one needle, in various instances two needles.

### The Output Units

The automatic water sampler apparatus 100, in particular each water collector has its own output unit (10.5, 20.5, 30.5, 40.5), which is configured to evacuate the water outside the apparatus, or to a waste disposal.

A check valve (10.8, 20.8, 30.8, 40.8) can be present upstream of the corresponding output unit (10.5, 20.5, 30.5, 40.5) and downstream of the corresponding 4-way stopcock (10.6, 20.6, 30.6, 40.6) in order to prevent that the evacuated water returns into the apparatus 100.

### The Main Controller Unit

In order to control the input unit, the dosing unit and the output unit, a main controller unit comprising a processor is present in the apparatus. The processor is usually a single-board computer. For example, the processor is a Raspberry Pi from the Raspberry Pi Foundation. The main controller unit can be managed in a remote way, for instance by wireless communication, so that the user can control the water sampler apparatus from the lab. The main controller unit is powered on a portable battery that is also part of the water sampler apparatus.

The portable battery can have a reloading system (e.g. a solar panel or a wind generator). To this end, the main controller unit ensures that stand-by energy demand is minimized by switching off the power of each device that is not in active operation.

One of the roles of the processor is to direct the sampling of water. The processor controls the sampling of water with regards to the number of water collectors connected to the water sampler apparatus. In the example when there are two water collectors (for example the precipitation and the stream water collectors), the processor is able to allows for sequential sampling (one collector after the other) or for simultaneous sampling (all collectors performing the sampling at the same time).

The processor can perform high-frequency (simultaneous) sampling with a maximum rate ranging between one sample per minute and one sample per hour. The process can obviously work with a lower rate, namely with a rate ranging between one sample per two hours and one sample per month. The main controller unit is also equipped with a communication unit, for instance a modem, allowing for remote control.

A user-friendly sampler interface permits defining a sampling scheme and interrogating the metadata related to the sampling. The water sampler can be connected to external sensors and dataloggers, and sampling operations can be triggered by sensor signals or measurements (e.g. water stage, water conductivity and/or signals from a precipitation sensor, as well as the precipitation gauge). The stored data can also be transferred to an external device using a portable transfer unit (e.g. USB stick).

### Method of Sampling

FIGS. 6a to 6i show a representation of the flow options of the 4-way stopcock 10.6. The flow is indicated by the plain arrows. For ease of representation, the representation on FIGS. 6a to 6i is also working for the 4-way stopcock 20.6, 30.6 and 40.6.

The following will describe the sequential sampling steps:

Step 1: On FIG. 6a, the liquid (e.g. the water) passes through the 4-way stopcock without being sucked by the corresponding dosing unit and without being directed/ejected to the vials. In fact, the liquid goes directly toward the output unit (e.g. 10.5). The corresponding pump 10.10 is thus switched on. By being directed towards the output unit, any the dead volume in the conduits of the apparatus is pumped out.

Step 2: On FIG. 6b, the dosing unit is activated: the dosing unit or the syringe (e.g. 10.2) sucks the liquid.

Step 3: On FIG. 6c, the water that has been sucked in the previous step is reinjected into the conduits in order to rinse the dosing unit or the syringe and flush back the filter (for instance, the filter 10.7 shown on FIG. 2). This configuration is adopted when the rinsing mode is activated.

The steps 2 and 3 can be optionally repeated in order to improve the rinsing of the system. For instance, those steps can be repeated three times. The number of times that the syringe is rinsed and the volume that it collects can vary and are to be specified by the main controller unit, directly to the interface of the main controller or via a remote control. The rinsing is thus performed with the water to be sampled, in order to minimize contamination and memory effects.

Step 4: Once the rinsing has been achieved, the pumping unit (or the syringe) sucks the water (FIG. 6d). The amount of water sucked corresponds to the full volume capacity of the syringe (typically 60 mL of water). The dosing volume which has to be sampled varies between 1 mL and 200 mL of water, in various instances between 2 mL and 40 mL. It can also be for instance 2 mL, 4 mL, 10 mL and 12 mL. A dead volume (ranging from 1 mL to 5 mL, in various instances amounting to 1.5 mL) corresponding to the volume of the tubing between the pumping unit and the needle needs to be considered.

Step 5: Then, the inflow is stopped, meaning that the main controller unit will turn off the pump 10.10.

Step 6: On FIG. 6e, the dosing unit pushes out through the injection unit (and the needle) the dead volume which is ejected into a trash reservoir or outside the sampler, in order to rinse the injection unit and the needle.

Step 7: The injection unit (and the needle) is moved exactly above the predetermined vial, in which the liquid has to be ejected. This is provided thanks to the two positioning units 106.

Step 8: On FIG. 6f, which is actually a similar position for the 4-way stopcock than in FIG. 6e, the dosing unit ejects the dosing volume of liquid (e.g. 1 mL, 2 mL, 4 mL, 10 mL, 12 mL or 40 mL) into the predetermined vial. The actual sampling is thus achieved. With regard to the vials, closures with pre-pierced septa can facilitate air removal while the sample is injected. These are manufactured by making a slit in the septa. Alternatively, a second needle in the injection unit can be used in order to release the excessive pressure. The second needle is then only in connection with the surrounding atmosphere.

Step 9: The injection unit (or the needle(s)) is thus removed from the vial.

Step 10: As indicated by the dashed arrow on FIG. 6g, the dosing unit sucks air after having delivered the liquid into the predetermined vial. This will allow for setting up the dosing unit in a position configured to empty the rest of liquid (in the next step) contained inside the dosing unit.

Step 11: The pump 10.10 is switched on again and the excess of the liquid is ejected from the dosing unit into an output unit (e.g. 10.5) (FIG. 6h).

Step 12: The liquid (the water) passes again through the 4-way stopcock without being sucked by the corresponding dosing unit and without being directed to the vials (FIG. 6i). The liquid goes directly toward the output unit.

The whole cycle (steps 1 to 12) can start again in order to sample water in another vial. Sampling is performed until an amount up to 1600 vials is filled. Afterwards, the tray 104 can be manually and/or automatically replaced by another tray in order to keep sampling, while the vials are analysed in the laboratory.

### Preservation and Memory Effects

The method used for sampling has a large impact on the quality of the collected samples. It is the main objective of an automatic water sampler apparatus to collect fully representative samples. To this end, contamination and cross-contamination of water samples should be minimised and samples should be preserved during the sampling period.

Memory effects refer to the impacts of antecedent samples on current sampling. In the presented automatic water sampler apparatus, memory effects can be avoided by rinsing the dosing and injection unit before collecting a new sample.

FIG. 7 represents the chloride concentration in a reference water sample and samples collected with the automatic water sampler apparatus (referred as “Sampled”) after no rinsing, after rinsing the dosing and injection units once and after rinsing them twice. It is clear on FIG. 7 that the sampling apparatus should be rinsed twice between two consecutive samples to avoid contamination and memory effects. It is only after rinsing the apparatus twice that the apparatus is able to collect samples fully comparable to the reference sample.

1.-21. (canceled)

22. An automatic water sampler apparatus comprising:
an input unit adapted to collect water, the input unit comprising:
at least one of:
a first water collector for precipitation, the first water collector comprising a precipitation gauge;
a second water collector for surface water;
a third water collector for groundwater;
a fourth water collector for soil water;
a pump per water collector;
one dosing unit per water collector;
one movable injection unit that comprises at least one needle per water collector;
one tray with a plurality of vials, each vial comprising a cap;
an output unit per water collector, adapted to evacuate the water outside the apparatus; and
a main controller unit adapted to control the input unit, the dosing unit, the movable injection unit and the output unit,
wherein each of the water collectors is independently in fluidic connection with the dosing unit and the movable injection unit,
wherein the dosing unit is in fluidic connection with the output unit, and
wherein the main controller unit comprises a processor configured to perform:
in the case where the input unit comprises one of the water collectors, sampling of the one water collector, or
in the case where the input unit comprises at least two of the water collectors, simultaneous sampling of the at least two of the water collectors.

23. The automatic water sampler apparatus according to claim 22, wherein the processor is configured to perform high-frequency sampling or high-frequency simultaneous sampling at a maximum rate ranging between one sample per minute and one sample per hour.

24. The automatic water sampler apparatus according to claim 22, wherein the processor is configured to perform sampling or simultaneous sampling at a rate ranging between one sample per minute and one sample per month.

25. The automatic water sampler apparatus according to claim 22, wherein the one dosing unit per water collector comprises a reciprocating pump.

26. The automatic water sampler apparatus according to claim 22, wherein the input unit comprises at least the first water collector for precipitation, the first water collector comprising:
a funnel adapted to collect precipitation;
a first closed container; and
a second closed container;
the first and second closed containers comprising respectively a first and a second air-release opening, a first and a second inlet and a first and a second outlet;
the first and second closed containers being fluidly connected to the one dosing unit respectively through the first and second outlets by means of a merging element; and
the funnel comprising a conduit, the conduit being fluidly connected to the first closed container through the first inlet.

27. The automatic water sampler apparatus according to claim 22, wherein the input unit comprises at least the first water collector for precipitation, the first water collector comprising:
a funnel adapted to collect precipitation;
a first closed container; and
a second closed container;
the first and second closed containers comprising respectively a first and a second air-release opening, a first and a second inlet and a first and a second outlet;
the first and second closed containers being fluidly connected to the one dosing unit respectively through the first and second outlets by means of a merging element; and
the funnel comprising a conduit, the conduit being fluidly connected to the second closed container through the second inlet.

28. The automatic water sampler apparatus according to claim 26, wherein the conduit is a flexible conduit.

29. The automatic water sampler apparatus according to claim 26, wherein the first and second closed containers are fluidly connected to the one dosing unit respectively through the first and second outlet through a first and second 3-way stopcock.

30. The automatic water sampler apparatus according to claim 29, wherein each of the first and second 3-way stopcocks comprises:
a first way which is respectively fluidly connected to the first outlet or the second outlet;
a second way which is fluidly connected to the one dosing unit by means of the merging element; and
a third way, the third way being respectively a first fluidic exit and a second fluidic exit.

31. The automatic water sampler apparatus according to claim 29, wherein the first and second 3-way stopcocks are electrically and/or mechanically connected to a first and second actuator, the first and second actuators comprising respectively a first and second control device.

32. The automatic water sampler apparatus according to claim 26, wherein the funnel comprises a removable water filter.

33. The automatic water sampler apparatus according to claim 26, wherein the first and second closed containers each has a volume up to 500 mL.

34. The automatic water sampler apparatus according to claim 22, wherein the fluidic connection between each of the water collectors and the corresponding dosing unit is a corresponding 4-way stopcock,
at least one of:
in the case where the input unit comprises at least the first water collector or precipitation, the 4-way stopcock comprises a first way which is fluidly connected to the merging element; and
in the case where the input unit comprises at least one of the second, third and fourth water collectors, the 4-way stopcock comprises a first way which is directly fluidly connected to the second, third or fourth water collectors;
the 4-way stopcock comprises a second way which is fluidly connected to the dosing unit;
the 4-way stopcock comprises a third way which is fluidly connected to the output unit; and
the 4-way stopcock comprises a fourth way which is fluidly connected to the movable injection unit.

35. The automatic water sampler apparatus according to claim 34, wherein the 4-way stopcock is at least one of electrically and mechanically connected to a third actuator, the third actuator comprising a third control device.

36. The automatic water sampler apparatus according to claim 22, wherein the movable injection unit comprises two needles.

37. The automatic water sampler apparatus according to claim 22, wherein the apparatus is fitted within a frame, the frame further comprising two positioning units configured to position the at least one movable injection unit to a predefined location of the tray.

38. The automatic water sampler apparatus according claim 22, wherein the automatic water sampler apparatus further comprises at least one portable battery configured to power the automatic water sample apparatus.

39. The automatic water sampler apparatus according to claim 22, wherein the processor is a single-board computer.

40. The automatic water sampler apparatus according to claim 22, wherein the input unit comprises any combination of at least one of:
at least one first water collector for precipitation;
at least one second water collector for surface water;
at least one third water collector for groundwater; and
at least one fourth water collector for soil water.
